(19) United States
(12) Patent Application Publication
(10) Pub. No.: US 2022/0322920 A1
(43) Pub. Date: Oct. 13, 2022

(54) Endoscope Steering Mechanism with Everted Tube Introducer

(71) Applicant: University of Washington, Seattle, WA (US)

(72) Inventors: Andrew Lewis, Seattle, WA (US); Blake Hannaford, Seattle, WA (US); Eric J. Seibel, Seattle, WA (US)

(21) Appl. No.: 17/595,946

(22) PCT Filed: Jun. 1, 2020
(86) PCT No.: PCT/US2020/035589

§ 371 Date: Nov. 30, 2021

Related U.S. Application Data
(60) Provisional application No. 62/854,696, filed on May 30, 2019.

(51) Int. Cl. A61B 1/00; A61B 1/05; A61B 1/045; A61B 1/005

(52) U.S. Cl. CPC A61B 1/00151; A61B 1/00154; A61B 1/0016; A61B 1/05; A61B 1/00006; A61B 1/045; A61B 1/00097; A61B 1/0057

(57) ABSTRACT

The present disclosure provides an introducer device including an outer tube defining a first lumen, and an inner tube defining a second lumen, and a sheath coupled thereto. The first end of the inner tube is positioned in the first lumen of the outer tube, and the inner tube is configured to translate within the first lumen in a direction towards the first end of the outer tube from a first position to a second position. The first end of the sheath is coupled to the first end of the outer tube, and the second end of the sheath is coupled to the first end of the inner tube. The introducer device further includes an inlet port in fluid communication with the inner surface of the outer tube, and one or more steering wires coupled to the sheath that are configured to bias a direction of a leading edge of the sheath as the sheath everts from a retracted position to an extended position.

### CROSS-REFERENCE TO RELATED APPLICATIONS

This application claims priority to U.S. Provisional Patent Application No. 62/854,696, filed May 30, 2019, the contents of which are hereby incorporated by reference in their entirety.

### BACKGROUND

Unless otherwise indicated herein, the materials described in this section are not prior art to the claims in this application and are not admitted to be prior art by inclusion in this section.

Bladder cancer requires ongoing screenings in order to identify recurrence in time for effective removal. These bladder scans, called cystoscopies, are performed by trained urologists in hospitals using rigid or flexible endoscopes inserted into the urethra. Multiple scopes may be used in order to ensure total screening coverage of the inside of the bladder. This insertion and movement within the bladder is often painful and uncomfortable, which, in addition to other factors, can lead to patients going further between cystoscopy procedures or stopping them altogether. This presents significant risk to the patient, as bladder cancer can be specifically aggressive if not caught early.

Regionalization of bladder cancer care has steadily increased over the past few decades, resulting in the majority of bladder cancer care occurring in urban centers. Even though a cystoscopy is a procedure that all urologists can perform outside of specialty centers, the changing demographics of urologic practices has made access to this procedure difficult for patients living in rural areas.

Therefore, an improved system and methods of use that enable the use of a teleoperated robot to perform cystoscopies in rural settings may be desirable. Performing cystoscopies in rural clinics will minimize the travel burden on patients, lower travel reimbursements for single-payer health systems, and free up clinical space and resources at urban hospitals.

### SUMMARY

Example devices and methods described herein describe an introducer device, systems including the introducer device, and methods for use. In particular, the present disclosure provides an introducer device that provides a working lumen or active guiding for another device (such as an endoscope) which may cause harm or discomfort without a guiding barrier or sheath.

Thus, in one aspect, an introducer device is provided including (a) an outer tube defining a first lumen and having a first end and a second end, (b) an inner tube defining a second lumen and having a first end and a second end, wherein the first end of the inner tube is positioned in the first lumen of the outer tube, and wherein the inner tube is configured to translate within the first lumen in a direction towards the first end of the outer tube from a first position to a second position, (c) a sheath having a first end and a second end, wherein the first end of the sheath is coupled to the first end of the outer tube, and wherein the second end of the sheath is coupled to the first end of the inner tube, (d) an inlet port in fluid communication with a space defined by an area between an outer surface of the inner tube, an inner surface of the outer tube, and the sheath, wherein the sheath is configured to evert from a retracted position within the first lumen of the outer tube to an extended position extending from the first end of the outer tube, and wherein the sheath everts from the retracted position to the extended position when a leading edge of the sheath is advanced past the first end of the outer tube by applying a fluid pressure to the space via the inlet port and simultaneously translating the inner tube from the first position to the second position, and (e) one or more steering wires coupled to the sheath, wherein the one or more steering wires are configured to bias a direction of the leading edge of the sheath as the sheath everts from the retracted position to the extended position.

In a second aspect, a system is provided. The system may include (a) the introducer device of the first aspect, (b) an elongated member positioned at least partially within a working lumen of the introducer device, wherein the working lumen of the introducer device is defined by a space between opposing walls of the everted sheath, (c) at least one processor, and (d) data storage including program instructions stored thereon that when executed by the at least one processor, cause the system to perform functions. The functions may include (i) apply, via the inlet port, fluid pressure to the space between the outer surface of the inner tube, the inner surface of the outer tube, and the sheath, (ii) translate the inner tube from the first position to the second position to thereby cause the sheath to evert from the retracted positioned within the first lumen of the outer tube to the extended position extending from the first end of the outer tube, and (iii) translate the elongated member through the working lumen in a direction towards the leading edge of the sheath.

In a third aspect, a method is provided. The method may include (a) positioning the introducer device of the first aspect in proximity to a body lumen of a patient, (b) applying, via the inlet port of the introducer device, fluid pressure to the space between the outer surface of the inner tube, the inner surface of the outer tube, and the sheath, and (c) translating the inner tube from the first position to the second position to thereby cause the sheath to evert from the retracted positioned within the first lumen of the outer tube to the extended position extending from the first end of the outer tube until the leading edge of the sheath reaches a target anatomy in the patient.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a simplified diagram of a teleoperation system, according to an example embodiment.

FIG. 1B is a simplified diagram of a teleoperation station for use with the teleoperation system of FIG. 1A, according to an example embodiment.

FIG. 2A is a side cross-sectional view of an introducer device in a first position, according to an example embodiment.

FIG. 2B is a side cross-sectional view of the introducer device of FIG. 2A in a second position, according to an example embodiment.

FIG. 3A is a detailed side cross-sectional view of the introducer device of FIG. 2A, according to an example embodiment.

FIG. 3B is a detailed side cross-sectional view of the introducer device of FIG. 3A with a fluid positioned therein, according to an example embodiment.

FIG. 4 is a detailed side cross-sectional view of the sheath of the introducer device with one or more steering wires, according to an example embodiment.

FIG. 5 is a perspective view of the introducer device, according to an example embodiment.

FIG. 6 is a cross-sectional view of the sheath of another example introducer device, according to an example embodiment.

FIG. 7 is a detailed side cross-sectional view of the end of the introducer device including the sheath, according to an example embodiment.

FIG. 8 is a detailed side cross-sectional view of an example sheath of the introducer device, according to an example embodiment.

FIG. 9 is a side cross-sectional view of the sheath as it everts when in use, according to an example embodiment.

FIG. 10 illustrates an everted sheath being introduced through the urethra towards the bladder of the patient, according to an example embodiment.

FIG. 11 is a side view of an example linear motion mechanism, according to an example embodiment.

FIG. 12 is a perspective view of the linear motion mechanism of FIG. 11, according to an example embodiment.

FIG. 13 is a perspective view of the introducer device coupled to the linear motion mechanism of FIG. 11, according to an example embodiment.

FIG. 14 illustrates a cover for the introducer device, according to an example embodiment.

FIG. 15 is a simplified block diagram of an example system, according to an example embodiment.

FIG. 16 illustrates an example elongated member, according to an example embodiment.

FIG. 17 illustrates an example ultrasonic probe, according to an example embodiment.

FIG. 18 illustrates shows an ultrasonic beam probe sensor and ultrasonic sphere probe sensor, according to an example embodiment.

FIG. 19 illustrates one or more bending slits in a distal section of the elongated member, according to an example embodiment.

FIG. 20 is a side view of the elongated member, according to an example embodiment.

FIG. 21 is a perspective view of a plurality of components of a second liner motion mechanism of the elongated member, according to an example embodiment.

FIG. 22 is a top view of the plurality of components of the second liner motion mechanism of FIG. 21, according to an example embodiment.

FIG. 23 illustrates an elongated member steering interface, according to an example embodiment.

FIG. 24 is a perspective view of the second linear motion mechanism, according to an example embodiment.

FIG. 25 is a perspective view of the second linear motion mechanism of FIG. 24 with the introducer device positioned thereon, according to an example embodiment.

FIG. 26 illustrates an alternate actuator layout and interface for elongated member, according to an example embodiment.

FIG. 27 illustrates an alternative approach with a commercial flexible endoscope mounted to an actuation platform integrated with the introducer module, according to an example embodiment.

FIG. 28 is a flowchart illustrating an example method according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

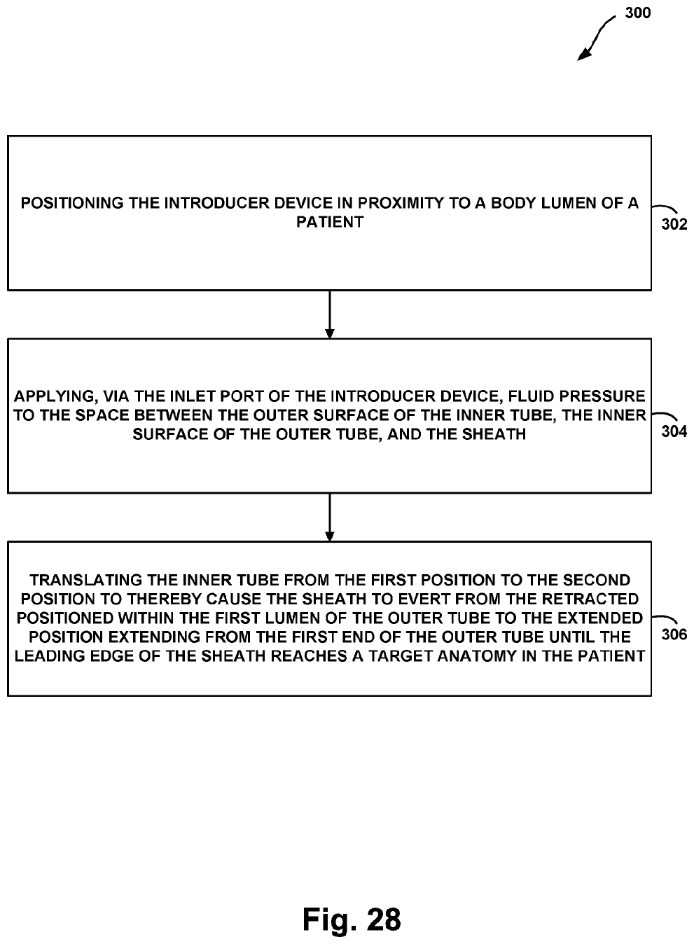

In FIG. 28, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 28 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

The present disclosure provides an introducer device and corresponding system that provides a working lumen for an endoscope (or other elongated device) to access a target anatomy of a patient. Although the focus of the present disclosure discusses uses in urology such as scanning a bladder of the patient via access through the urethra, the devices and systems described herein may be used in any body lumen to access any target anatomy inside of the patient.

Thus, in one particular example, the present disclosure provides a bladder scanning system that may be teleoperated by remote experts and set up by nurses. A soft, balloon-like sheath of an introducer device of the system is pressurized and allowed to evert (roll from the inside) into the urethra in order to act as a protective sheath for a flexible endoscope to gently travel therethrough. The flexible endoscope can provide feedback about the sheath insertion as it everts along the urethra without sliding or the endoscope can be inserted after the sheath has reached the opening of the bladder. Once inside the bladder, the flexible endoscope is controlled by the doctor locally, remotely, or autonomously to inspect the inside surface of the bladder. Other associated technologies may be attached to or inserted through the soft sheath or endoscope to provide interventional capabilities in order to further diagnose or treat abnormalities found in the initial scan. Any of these functions could be performed manually, semi-autonomously, autonomously, or via teleoperation.

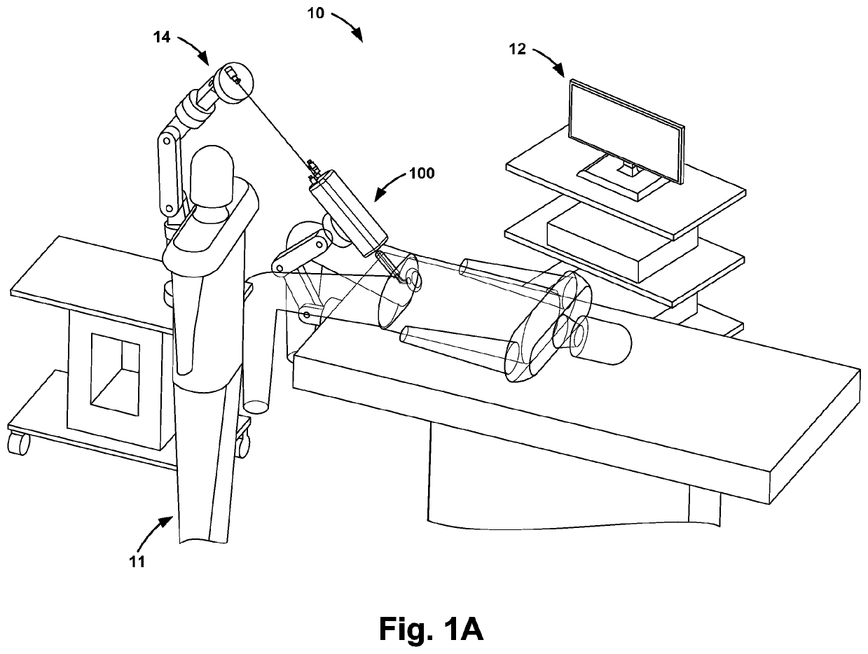

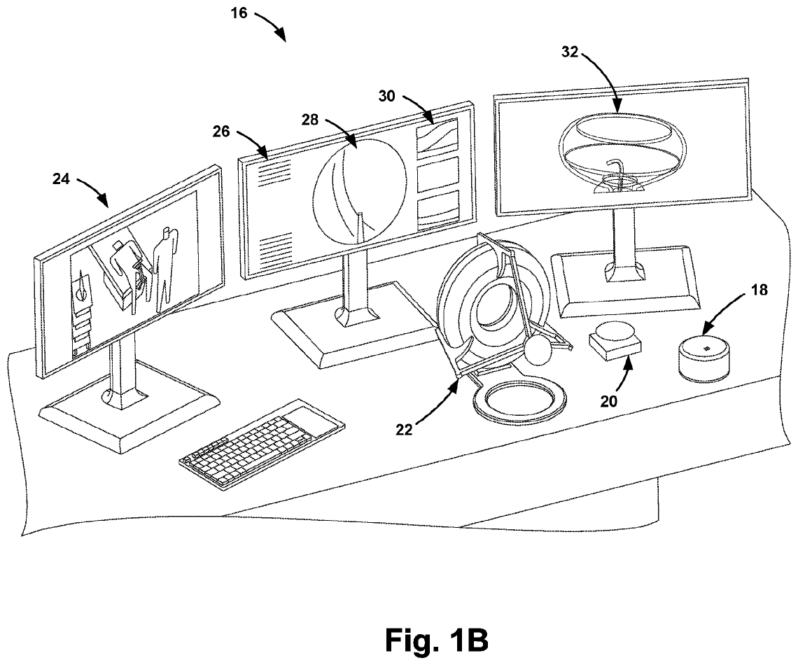

In the example of a teleoperation system 10 is illustrated in FIG. 1A. Such a teleoperation system 10 could provide outpatient care to rural communities while still giving patients access to expert urologists that tend to reside at urban hospitals. As shown in FIG. 1A, the teleoperation system 10 described herein is designed to be set up and overseen by nurses or technicians 11 at the patient site. A monitor 12 is provided to monitor the procedure and to communicate with the urologist who is located elsewhere. The teleoperation system 10 may further include a robotic system 14 that can be remotely operated by the urologist. In use, the soft, flexible approach of the introducer device 100 described herein may reduce discomfort during and after the procedure. Together, these improvements could lead to increased adherence to cystoscopy schedules.

It is also possible that such a platform could be automated to perform full-coverage cystoscopy scans and send a 2D- or 3D-scan of the bladder to an expert urologist or oncologist. The scan may also be analyzed by an artificial intelligence (AI) system in order to highlight specific abnormalities to be identified or investigated by the appropriate specialist, either through normal RGB image analysis, alternative wavelength analysis, or some combination with biomarkers or other cancer identifying techniques. The specialist may wish to teleoperate the robot in order to investigate further, or approve the analysis of the robot AI, which could then be equipped with interventional technologies for teleoperated or autonomous intervention.

Any aspect of the present disclosure could be designed to be controlled manually. This could allow the nurse or technician to operate some aspects of the platform while the rest of the platform is teleoperated. Or it could allow for the improvements to patient comfort to be enabled at a cost lower than a fully robotic system. Both the component cost and regulatory cost of a partially- or fully-manual system could be expected to be lower.

Manual or autonomous mosaic scans/3D models of the bladder will allow for tracking of the robotic tip for ease of visualization by the surgeon and for added safety in the control of the robot. Safety will be ensured with redundancy in the sensing and actuation systems. Machine vision can be used to ensure separation between the camera tip and the anatomy. Further, flexible insertion and actuation will ensure minimal interaction forces between the robot and the anatomy. In addition, sensing of bending with bend sensors and actuator sensing will be combined with knowledge of the anatomy (from the 3D model) to ensure minimal accidental interactions with anatomy. The 3D model can be used with the robotic system 14 to ensure proper placement of therapies that may be used in conjunction with the teleoperation system 10. Further, such external platforms can also be given a safety range of positions that maintain the working tip at a specific point in the bladder or within the visual range of this platform's endoscope.

The surgeon will have a teleoperation station at the home hospital or clinic at which they practice. This station will have an internet link to the hospital or clinic with the robotic system 14. Robot commands from the human interface hardware and software will be sent to the robotic system 14 over the internet connection, and the endoscopic camera feed, robot sensing and control data, external imaging data, and operating room camera feeds will be among the data sent back to the operating station. Robotic control data will be combined with endoscopic mosaic models to provide to the surgeon a 3D representation of the bladder anatomy and the location and shape of the robotic system 14.

These representations will also be used as part of a safety system on both sides of the network connection. The possibility of time delay in the communication requires that the robotic system 14 be able to decide whether to execute commands sent to it by the surgeon, limit the requested actions, or ignore the actions and communicate those limitations to the surgeon appropriately.

The surgeon may have access to a high-level automation interface. This software would allow the surgeon to pick from a variety of autonomous behaviors for the robotic system 14 to perform. The robotic system 14 will then either perform the action based on sensor data and pre-operative data, or it will request additional information from the surgeon, such as specific regions of interest, approach angles, or other behavior parameters. The software may present a simulated plan for the surgeon to approve before execution. The surgeon will be able to revise the plan and stop or alter the plan during execution. The robotic system 14 will be able to update its behavior based on data sensed during execution, but will be provided with safety limits that will limit or stop execution if the actual behavior deviates too far from the approved plan.

FIG. 1B shows an example teleoperation station 16 for use with the teleoperation system 10 of FIG. 1A. As shown in FIG. 1B, the teleoperation station 16 includes a voice transcribing assistant 18, an emergency stop button 20, a 3D controller interface 22, and screens with: live Operating Room overview 24; live cystoscope video 26, system data 28, extra sensor data 30; and virtual representation 32 of the prior scanned bladder with updates or comparisons with data from the current scan. Voice transcriptions will be added to records of the procedure or used to mark areas of interest on the virtual representation which can then be used for automatic targeting by the robot for reinspection or automatic therapy.

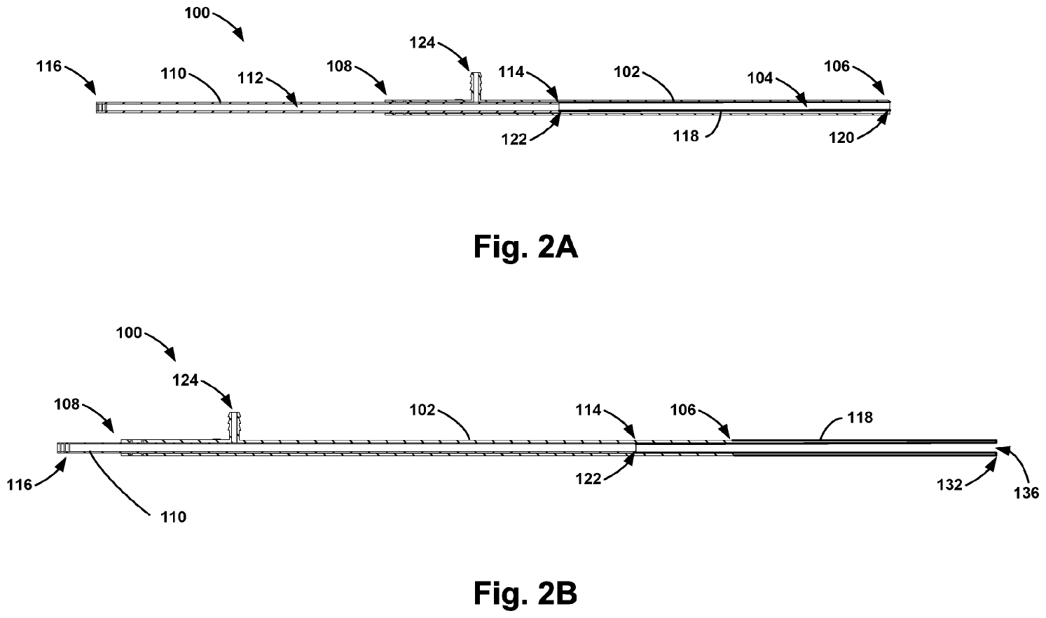

FIGS. 2A-2B illustrate a cross-sectional view of an introducer device 100 according to an example embodiment that may be used in the systems of FIGS. 1A-1B. As shown in FIGS. 2A-2B, the introducer device 100 includes an outer tube 102 defining a first lumen 104 and having a first end 106 and a second end 108. The introducer device 100 further includes an inner tube 110 defining a second lumen 112 and having a first end 114 and a second end 116. The first end 114 of the inner tube 110 is positioned in the first lumen 104 of the outer tube 102, and the inner tube 110 is configured to translate within the first lumen 104 in a direction towards the first end 106 of the outer tube 102 from a first position (shown in FIG. 2A) to a second position (shown in FIG. 2B). As shown in FIGS. 2A-2B, the introducer device 100 further includes a sheath 118 having a first end 120 and a second end 122. The first end 120 of the sheath 118 is coupled to the first end 106 of the outer tube 102, and the second end 122 of the sheath 118 is coupled to the first end 114 of the inner tube 110.

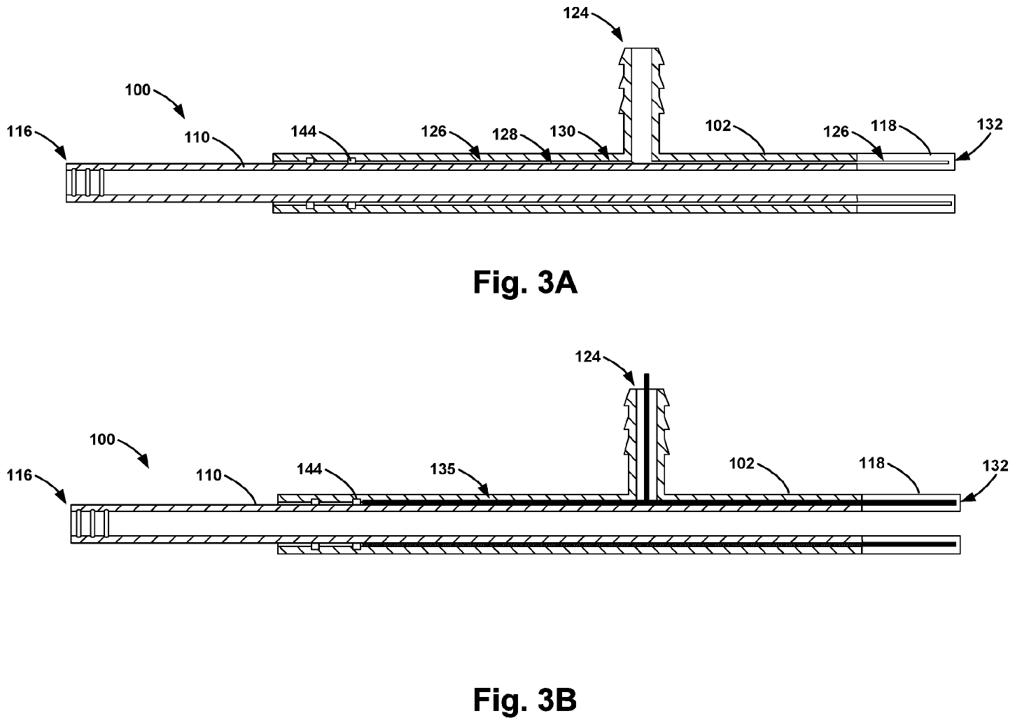

As shown in the detailed cross-sectional view of FIGS. 3A-3B, the introducer device 100 further includes an inlet port 124 in fluid communication with a space 126 defined by an area between an outer surface 128 of the inner tube 110, an inner surface 130 of the outer tube 102, and the sheath 118. The sheath 118 is configured to evert from a retracted position within the first lumen 104 of the outer tube 102 (shown in FIG. 2A) to an extended position extending from the first end 106 of the outer tube 102 (shown in FIG. 2B). The sheath 118 everts from the retracted position to the extended position when a leading edge 132 of the sheath 118 is advanced past the first end 106 of the outer tube 102 by applying a fluid pressure to the space 126 via the inlet port 124 and simultaneously translating the inner tube 110 from the first position to the second position. As shown in FIGS. 3A-3B, the introducer device 100 may include a fluid seal 144 extending from the outer surface 128 of the inner tube 110 to the inner surface 130 of the outer tube 102. The fluid seal 144 is positioned between the inlet port 124 and the second end 108 of the outer tube 102. The fluid seal 144 provides a sliding seal against the inner tube 110. Such a fluid seal 144 may be a disposable module to allow for the main body of the outer tube 102 to reusable. FIG. 3B illustrates a fluid 135 provided in the space 126 via the inlet port 124.

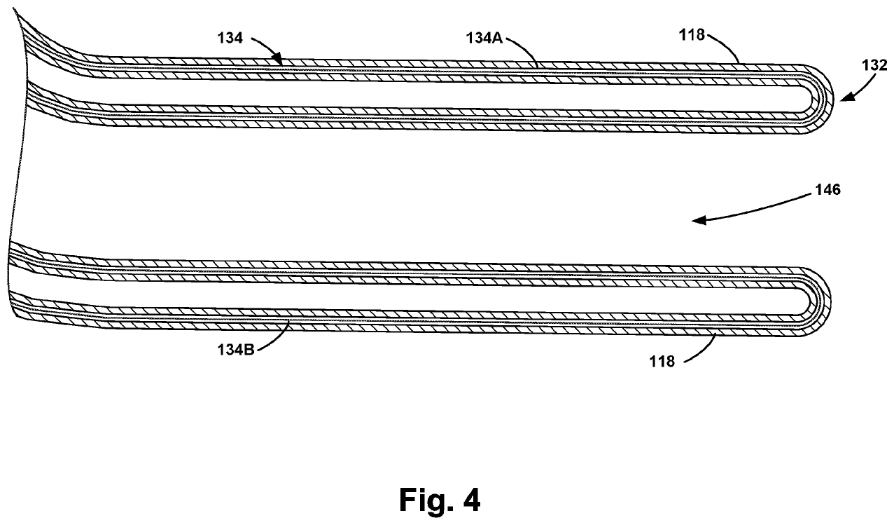

As shown in the detailed cross-sectional view of FIG. 4, the introducer device 100 further includes one or more steering wires 134 coupled to the sheath 118. In use, the one or more steering wires 134 are configured to bias a direction of the leading edge 132 of the sheath 118 as the sheath 118 everts from the retracted position to the extended position. In one particular example, the one or more steering wires 134 are embedded within the sheath 118. In another example, as shown in FIG. 4, the sheath 118 comprises an inner layer 119 and an outer layer 121, and the one or more steering wires 134 are positioned between the inner layer 119 of the sheath 118 and the outer layer 121 of the sheath 118. In such an example, a lubricant or a surfactant may be positioned between the inner layer 119 of the sheath 118 and the outer layer 121 of the sheath 118. As further shown in FIG. 4, the one or more steering wires 134 may comprise a first steering wire 134A positioned on a first side of the sheath 118 and a second steering wire 134B positioned on a second side of the sheath 118 opposite the first side. In such an example, the first steering wire 134A may be positioned approximately 180 degrees from the second steering wire 134B. Such an arrangement enables single-axis bending of the sheath 118 as the sheath 118 everts from the retracted position to the extended position. In another example, the one or more steering wires 134 may further include a third steering wire positioned approximately 90 degrees from the first steering wire 134A and the second steering wire 134B, and a fourth steering wire positioned approximately 180 degrees from the third steering wiring. Such an arrangement enables two-axis bending of the 118 as the sheath 118 everts from the retracted position to the extended position.

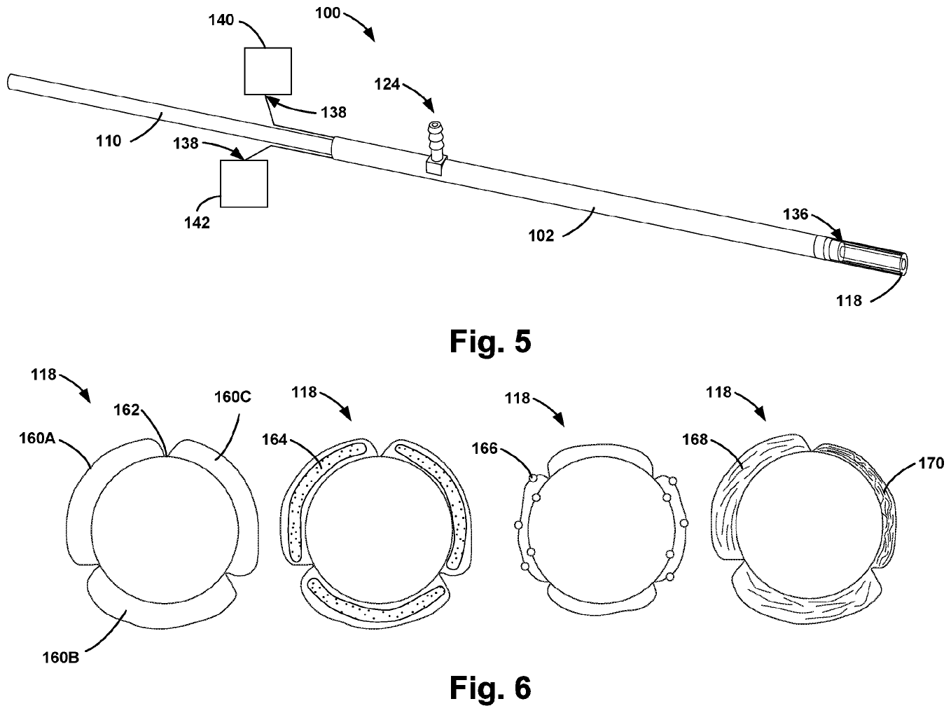

As shown in FIG. 5, each of the one or more steering wires 134 include a first end 136 and a second end 138. The first end of each of the of the one or more steering wires is coupled to the first end of the inner tube, and the second end of each of the one or more steering wires extends out of the first lumen 104 of the outer tube 102. In such an example, the introducer device 100 may include one or more motors 140 coupled to the one or more steering wires 134, such that activation of the one or more motors 140 adjusts a length of the one or more steering wires 134 to thereby bias the direction of the leading edge 132 of the sheath 118 as the sheath 118 everts from the retracted position to the extended position. In another example, the introducer device 100 may include one or more electromechanical brakes 142 coupled to the one or more steering wires 134, such that activation of the one or more brakes 142 adjusts a length of the one or more steering wires 134 to thereby bias the direction of the leading edge 132 of the sheath 118 as the sheath 118 everts from the retracted position to the extended position.

Thus, steering of the everted tube could be effected by the one or more steering wires 134 positioned in the sheath 118. Pulling on a steering wire positioned on one side of the introducer device 100 during eversion could bias the leading edge 132 of the sheath 118 towards going towards specific areas in anatomy that does not have one prescribed path. In open areas such as the insufflated bowel, the one or more steering wires 134 could be used to direct an endoscope for scanning or a tool for interventions. As shown in FIG. 5, the one or more steering wires could comprise individual cables which could be pulled (one pulled in and one let out) to bend the sheath 118 or bias the eversion of the sheath 118 up or down in the plane of the image.

An additional method for influencing the direction of the everted sheath 118 is illustrated in FIG. 6. As shown in FIG. 6, the sheath 118 may include individually controlled lengthwise pressure-sections 160A-160C built into the construction of the sheath 118. The simplest construction would introduce two sections into the sheath 118, while each one would have its own pressure supply and controller. In particular, the sheath 118 includes a first pressure section 160A in fluid communication with the inlet port 124, and the sheath further includes a second pressure section 160B in fluid communication with a second inlet port such that a pressure in the first pressure section 160A of the sheath 118 can be different than a pressure in the second pressure section 160B of the sheath 118 to thereby further bias the direction of the leading edge 132 of the sheath 118 as the sheath 118 everts from the retracted position to the extended position. This would allow for biasing of the sheath 118 to bend in the direction of the lower pressure side while everting the sheath 118. This may help avoid occlusions in a pathway or allow for finer control of opening constricted sections of the pathway.

More pressure sections would increase the resolution of directional control/influence of the sheath 118 during eversion. Sections may also be perforated once near the middle of the length so that the section can be used for inflation or suction. The perforations 164 can also be cut along the entire length or specific sections. Sections in the sheath 118 may be constructed from two tubes with a seal 162 running lengthwise to form the edge of each section. An additional flexible tube 166 may be inserted into the sections made by the seals. Stiffness of the everted sheath 118 may be useful when navigating the urethra or once the tube has been fully inserted and the surgeon is scanning or operating inside the bladder.

A possible stiffening mechanism is a sealed chamber (around the entire circumference or in individual sections) with loose, flexible ribbons 168 or strings that may have relatively high friction with each other where the section can be pressurized positively for free motion of the section, or pressurized negatively 170 to force the ribbons together, increasing internal friction and force dissipation area of that section. Instead of or in addition to ribbons, small particles (like coffee grounds) may be used for section stiffening. In such an example, when pressurized the sections are flexible, but when a vacuum is applied the friction between particles prevents motion to thereby stiffen that section. Some of these sections may have bend sensors integrated, or separate sections may be made just for bend sensors. Any of these section types may be used in combination with the others in any number of total sections. Pressurized sections may need additional valves or one-way valves to allow air or water to escape with initially pressurized.

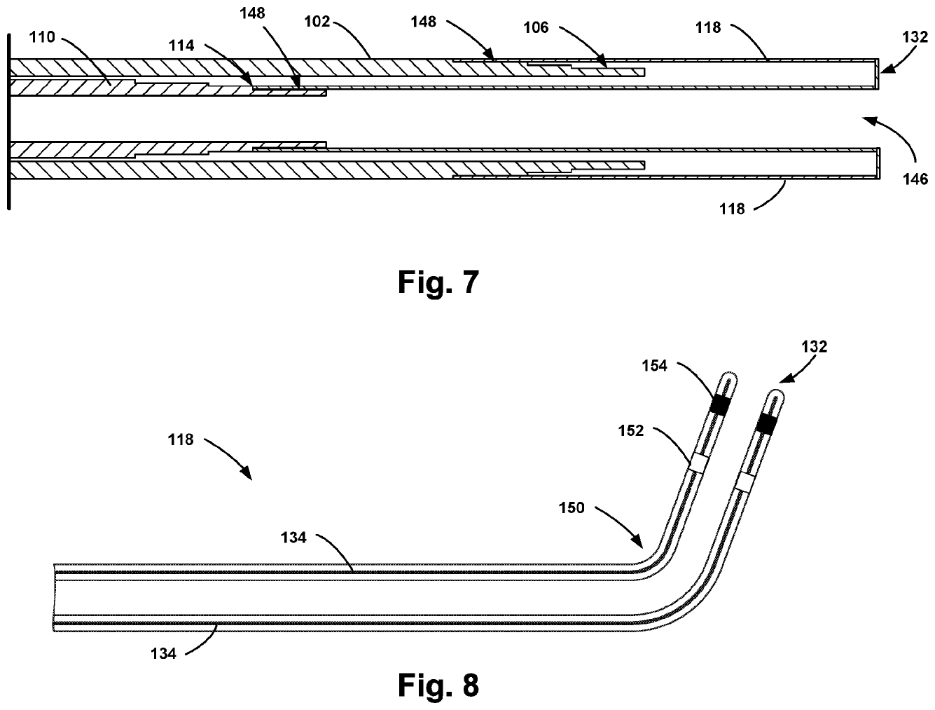

The introducer device 100 described herein provides a working lumen for an endoscope or some other device which may cause harm or discomfort without a guiding barrier or sheath. As shown in FIG. 7, a space between opposing walls of the everted sheath 118 define such a working lumen 146. The sheath 118 described herein is a watertight membrane which folds in on itself to make a tube to be everted into the urethra or other body lumen. During manufacturing, the sheath 118, the inner tube 110, and the outer tube 102 may be sealed together at the interfaces 148 shown in FIG. 7 and packaged as a sterile unit. Alternately the sheath 118 may be pre-sterilized with disposable sealing mechanisms that interface with the inner tube 110 and the outer tube 102 such that the inner tube 110 and the outer tube 102 may be reusable (after on-site sterilization). Finally, a combination of these approaches could be used where one of the tubes is disposable and the sheath 118 is pre-sealed to the disposable tube or attaches to both tubes just before use.

In one example, an elasticity of the sheath 118 is constant along its length. In another example, an elasticity of the sheath 118 is variable along its length. In one such example, the variability of the elasticity of the sheath 118 along its length is due to a variability of a thickness of the sheath along its length. Varying the elasticity of the sheath 118 along its length may enable the sheath to be tailored to a particular anatomy where it is known that certain areas require additional flexibility and compliance for the sheath 118. Additionally or alternatively, in one example the inner tube 110 and the outer tube 102 comprise a rigid material, and wherein the sheath 118 comprises a compliant material. In another example, a first portion of the outer tube 102 including the first end 106 has a first stiffness, and a second portion of the outer tube 102 including the second end 108 has a second stiffness that is greater than the first stiffness. Such an arrangement may be beneficial since the first end 106 of the outer tube 102 may interface with the patient, and thus more compliance on that end of the introducer device 100 may be beneficial for patient comfort.

In one example, as shown in FIG. 8, the sheath 118 includes one or more pre-formed curvatures 150 along the length of the sheath 118 as the sheath 118 transitions from the retracted positioned to the extended position. Such pre-formed curvatures 150 may be created for specific anatomies so that the sheath 118 more easily navigates the specific anatomy in which the introducer device 100 is intended to be used. In one particular example, as shown in FIG. 8, the pre-formed curvature 150 is positioned near the distal end of the sheath 118, and has an angle between about 90 degrees and about 150 degrees.

As shown in FIG. 8, the introducer device 100 may further include a tracking mechanism 152 embedded in the sheath 118 to track a position of the leading edge 132 of the sheath 118 as the sheath 118 everts from the retracted position to the extended position. Such a tracking mechanism 152 better enables the operator to track insertion progress through the patient's anatomy without requiring that an endoscope be inserted with the sheath 118. Additionally or alternatively, a contrast agent may be added to the pressurized fluid in the space 126 defined by the area between the outer surface 128 of the inner tube 110, the inner surface 130 of the outer tube 102, and the sheath 118. Such a contrast agent may similarly enables the operator to track insertion progress through the patient's anatomy without requiring that an endoscope be inserted with the sheath 118. The introducer device 100 may further include one or more sensors 154 positioned in the sheath 118. The one or more sensors 154 may further provide position information of the sheath 118 as the sheath 118 everts from the retracted position to the extended position. Further, the one or more sensors 154 may be configured to determine a pressure in the space 126 between the outer surface 128 of the inner tube 110, the inner surface 130 of the outer tube 102, and the sheath 118. Such pressure information may be used by the introducer device 100 to adjust a pressure of the fluid within the space 126 during eversion of the sheath 118.

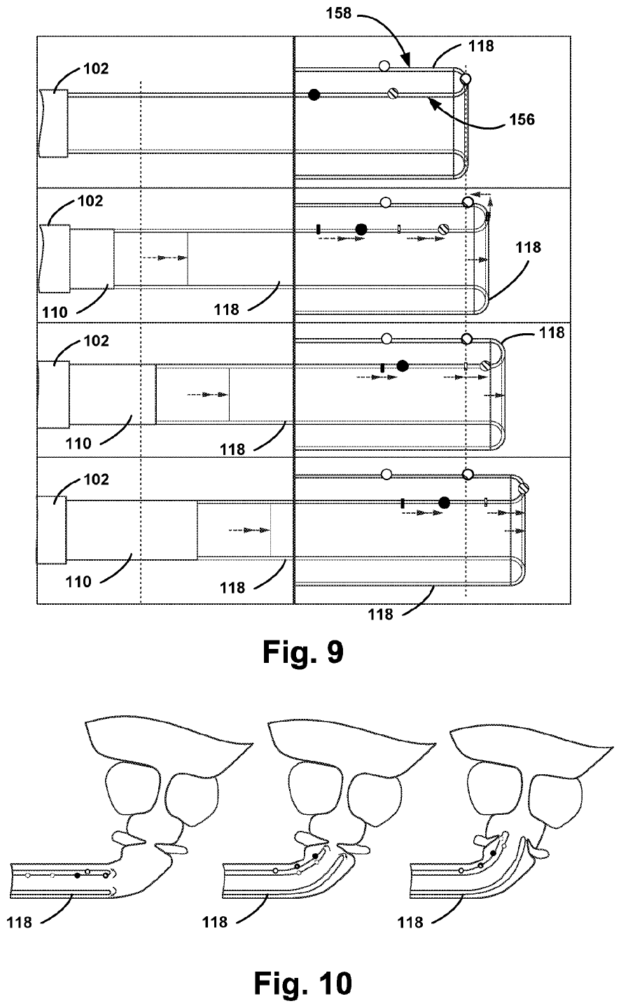

As discussed above, and as shown in FIG. 9, in use the inner tube 110 translates within the first lumen 104 in a direction towards the first end 106 of the outer tube 102 from a first position to a second position. Simultaneously, the leading edge 132 of the sheath 118 is advanced past the first end 106 of the outer tube 102 by applying a fluid pressure to the space via the inlet port to thereby evert the inner face 156 of the sheath 118 past the leading edge 132 and such that the inner face 156 everts to become the outside face 158 of a longer tube. Pressurized fluid (water or saline) or gas from the inlet port 124 provides a force against the leading edge 132 of the sheath 118 which causes the everting sheath to “grow” into the area of least resistance. This pressure also provides even force on constrictions within the urethra (or other body lumen), such as the bend present in most male anatomy and constrictions caused by enlarged prostates. The pressure within the space 126 can be controlled during procedures in order to cause minimum displacement of the urethra while ensuring that a sufficient channel is provided. An external source will provide the fluid or gas pressure to the introducer device 100 via the inlet port 124, and the external source may have its own sensing and control for pressure or volume, or the pressure and volume sensing and control may be integrated into the robotic platform of the introducer device 100. FIG. 10 illustrates the everted sheath 118 being introduced through the urethra towards the bladder of the patient.

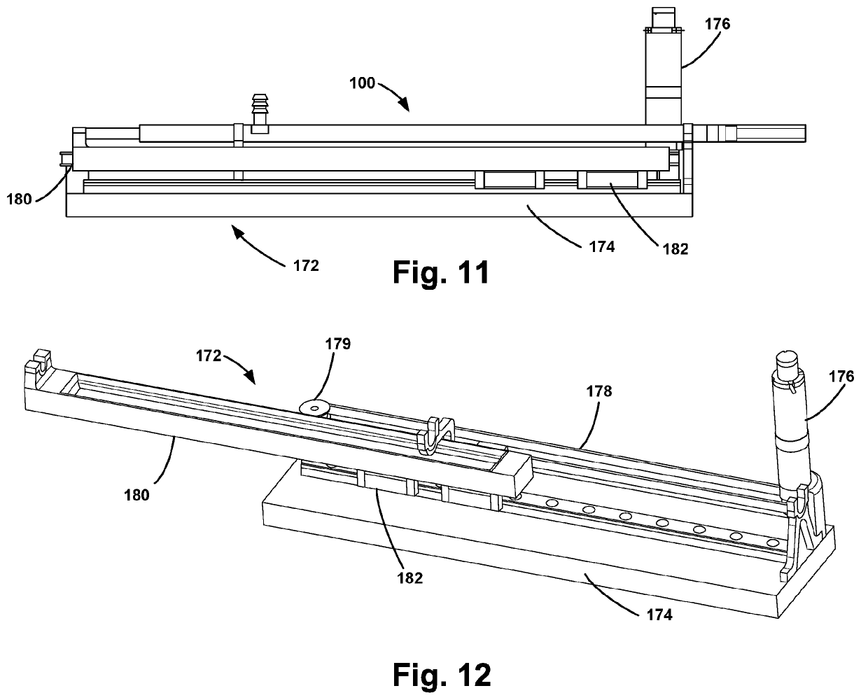

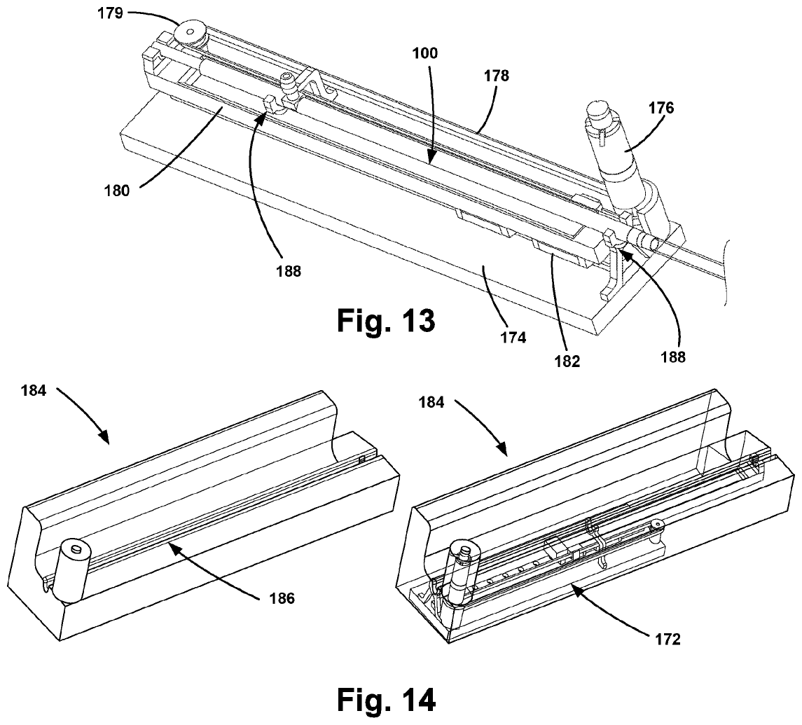

As shown in FIGS. 11-12, eversion of the sheath 118 may occur with the help of a linear motion mechanism 172 that translates the inner tube 110 within the first lumen 104 of the outer tube 102 in the direction towards the first end 106 of the outer tube 102 from the first position to the second position. FIG. 11 illustrates a side view of an example linear motion mechanism 172, while FIG. 12 illustrates a perspective view of the example linear motion mechanism 172. In the particular example illustrated in FIGS. 11-12, the linear motion mechanism 172 includes a base plate 174, a motor 176 coupled to the base plate 174, and a timing belt 178 coupled to the motor 176 with a corresponding timing belt pulley 179. Although a timing belt 178 is utilized in the embodiment of FIGS. 11-12, other linear actuators could be used such as a ball screw, a lead screw, a cable, or a linear motor as non-limiting examples.

The linear motion mechanism 172 further includes an inner tube mount 180 coupled to the inner tube 110, and the timing belt 178 is coupled to the inner tube mount 180 such that motion of the timing belt 178 is translated into linear motion of the inner tube 110. The linear motion mechanism 172 further includes a linear bearing 182 positioned between the inner tube mount 180 and the base plate 174. As such, the inner tube mount 180 is supported on the base plate 174 by the linear bearing 182 and the base plate 174 also supports the outer tube 102, while the inner tube 110 is only supported by the inner tube mount 180 and the sliding interface of the outer tube 102. FIG. 13 is a perspective view of the introducer device 100 coupled to the linear motion mechanism 172 of FIGS. 11-12, according to an example embodiment. Other possible linear actuators that could be used include acme or lead screws, ball screws linear motors, rack and pinion gears, or, for MRI compatibility, pneumatic linear steppers or rotary steppers with any of the mentioned rotary-to-linear motion mechanisms mentioned.

In one example, the introducer device 100 further includes a cover 184 removably positioned over each of the base plate 174, the motor 176, the timing belt 178, the inner tube mount 180, and the linear bearing 182. The cover 184 includes a slot 186 configured to receive the outer tube 102. As such, the cover 184 provides a sterile drape that separates the actuation mechanisms from the introducer device 100 and any device positioned within the working lumen 146 of the introducer device 100. The cover 184 maintains sterility for the components that contact the patient while allowing the more complicated mechanisms to stay clean-but-not-sterile. The cover 184 for this platform has integrated features that allow fastening to the actuator platform on the non-sterile side and to the sterilized components on the sterile side. FIG. 13 shows the linear motion mechanism 172 with interface points 188 affixed thereto, while the cover 184 which would be adhered to the perimeter of each of these features can be seen in FIG. 14. An enclosure for the platform (not shown) will contain the mechanical components, prevent drape tangling, and improve aesthetic aspects of the device. After installing the interface points 188 to the actuation components, the perimeter of the cover 184 will be gathered and affixed underneath the body of the platform and around the electronic power and communication cables of the platform. Thus, the platform will be isolated from the sterile environment. At this point, the introducer device 100 will be affixed to the cover 184 via the slot 186.

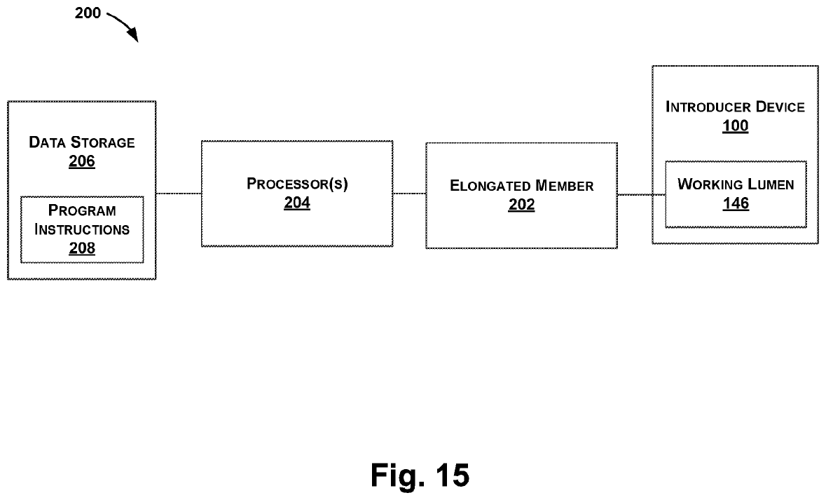

FIG. 15 illustrates an example system 200, according to an example embodiment. As shown in FIG. 15, the system 200 may include the introducer device 100 as described in FIGS. 1A-14, an elongated member 202 positioned at least partially within a working lumen 146 of the introducer device 100, at least one processor 204, and data storage 206 including program instructions 208 stored thereon that when executed by the at least one processor 204, cause the system 200 to perform functions.

In one example, the functions include (i) apply, via the inlet port 124, fluid pressure to the space 126 between the outer surface 128 of the inner tube 110, the inner surface 130 of the outer tube 102, and the sheath 118, (ii) translate the inner tube 110 from the first position to the second position to thereby cause the sheath 118 to evert from the retracted positioned within the first lumen 104 of the outer tube 102 to the extended position extending from the first end 106 of the outer tube 102, and (iii) translate the elongated member 202 through the working lumen 146 in a direction towards the leading edge 132 of the sheath 118. As discussed above, each of the introducer device 100 and the elongated member 202 may be configured for remote, semi-automated, or fully automated operation.

In one example, the program instructions 208 are further executable by the at least one processor 204 to cause the system 200 to (i) determine, based on one or more sensors 154 positioned in the sheath 118, a pressure in the space 126 between the outer surface 128 of the inner tube 110, the inner surface 130 of the outer tube 102, and the sheath 118, and (ii) adjust, based on the determined pressure, the fluid pressure applied to the space 126.

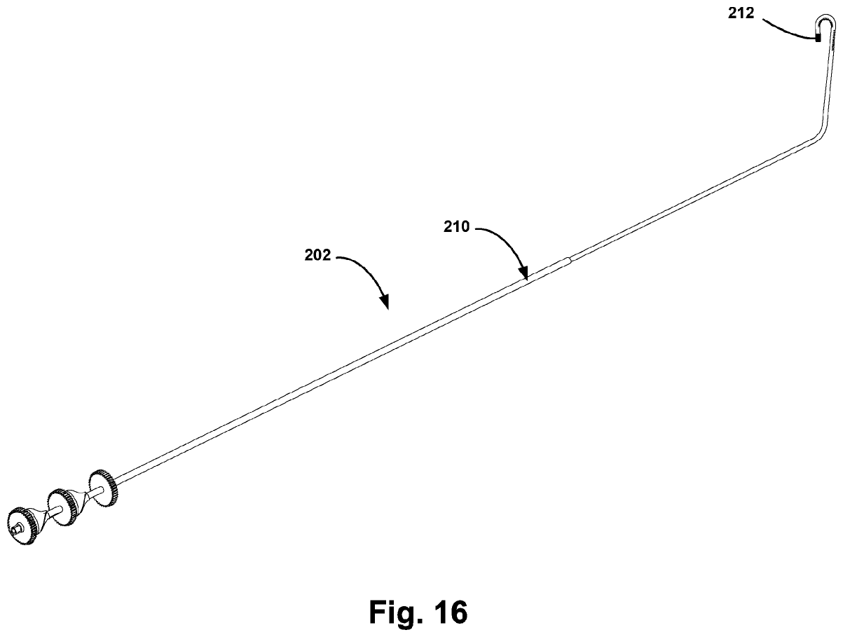

The elongated member 202 may comprise any tool or device that can utilize the working lumen 146 created by the introducer device 100 to access a target anatomy. In one example, as shown in FIG. 16, the elongated member 202 comprises an endoscope 210 including a camera 212. In such an example, the program instructions 208 may be further executable by the at least one processor 204 to cause the system 200 to (i) capture, via the camera 212 of the endoscope 210, one or more images of an anatomy into which the sheath 118 of the introducer device 100 is positioned, and (ii) based on the captured one or more images, bias the direction of the leading edge 132 of the sheath 118 via the one or more steering wires 134 coupled to the sheath 118 as the sheath 118 everts from the retracted position to the extended position to direct the leading edge 132 of the sheath 118 to a target anatomy. Such an arrangement provides a proactive approach to guiding the introducer device 100 to the target anatomy.

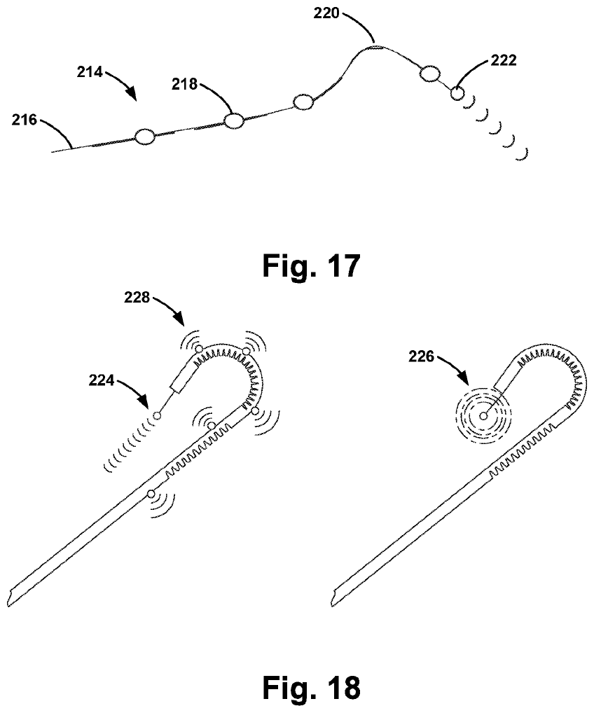

In another example, as shown in FIG. 17, the elongated member 202 comprises an ultrasonic probe 214 that provides information about the position of the sheath 118 of the introducer device 100 and/or the endoscope 210 within the bladder. In one particular example, the ultrasonic probe 214 includes a main wire 216 that may be embedded with one or more inflatable balloons 218 that provide friction against the working lumen 146 when inflated, thus maintaining the sensor's position relative to the end of the device. The main wire 216 may also have one or more bend sensing segments 220 (such as fiber optic-based shape sensing) attached, allowing for the ultrasonic probe 214 to provide shape sensing for the scope that it is working inside. Finally, on the tip of the main wire will be the ultrasonic transceiver 222, either in a beam or spherical configuration.

In another example, as shown in FIG. 18, an ultrasonic beam probe sensor 224 and/or an ultrasonic sphere probe sensor 226 can be used to add distance information to images and data about the closest feature to the tip, respectively. Such a probe can be inserted into a working channel in the elongated member 202 or sheath 118 or attached to the outside of either. An individual ultrasonic sensor 228 or an array of individually controlled sensors may be attached to the outside of the elongated member 202 or sheath 118 in order to estimate the shape of the elongated member 202 or sheath 118 or for mapping the shape of the bladder. When multiple sensors are used, they may be used in conjunction to gain additional 3D data about the flexible element and the bladder. The external sensors may be glued on to the needle or scope, taped temporarily, or embedded into a flexible removable sleeve that is affixed prior to operation.

In use, the elongated member 202 (e.g., the endoscope 210 and camera 212) are guided through the working lumen 146 of the introducer devices and provides actuation for scanning inside the bladder. The entire length of the elongated member 202 may be a flexible material, like Nitinol, or just the distal section roughly the length of the urethra may be flexible. This shorter flexible section would be bonded to a stiffer metal or plastic proximal section to transfer insertion and twisting forces. Flexibility of the elongated member 202 allows for bending around the bend of the urethra in male anatomy and also enables directional bending.

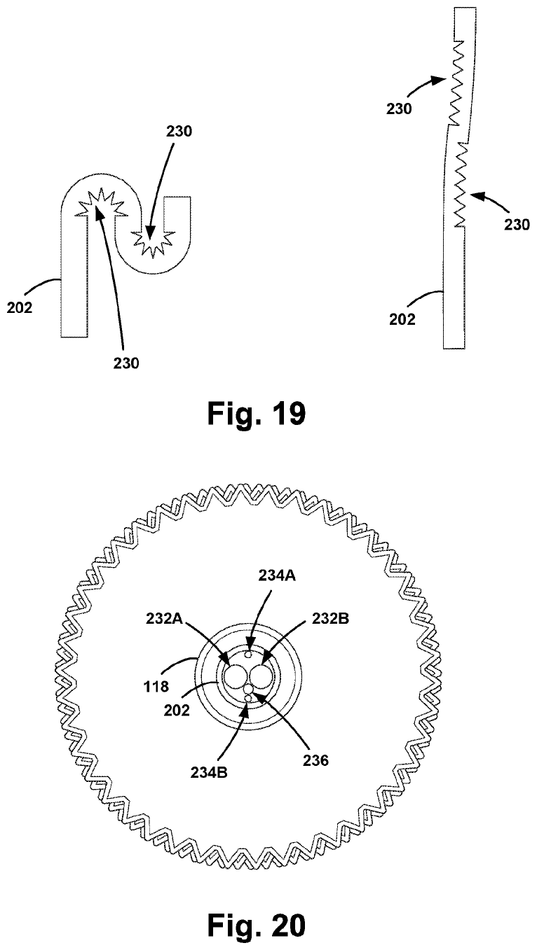

In one example, as shown in FIG. 19, the elongated member 202 includes one or more bending slits 230 in a distal section of the tubing. A cable (not shown) attached to the end of the distal part of the slit section 230 can be pulled to actuate the bending of the elongated member 202 in the direction of the slits 230. Multiple slit sections may be cut along the needle in order to affect a wider range of kinematics. FIG. 19 shows how two opposing slits can be independently actuated to achieve more complicated configurations than a single bend. Such configurations may be useful for irregular bladder anatomies or interventional approaches that require specific approach configurations. Twisting and insertion of the needle provide the final degrees of freedom (DOF) needed to scan the inner surface of the bladder.

The video feed from the camera 212 of the endoscope 210 may have visual overlays that indicate where the bending segments are in relation to the video. Further, there may be overlays that indicate the estimated shape of the elongated member 202 during operation. Teleoperation may be operated with any of the following or combinations thereof: haptic interfaces with 3 or more degrees of freedom; a sensorized model resembling the kinematics of the needle that can be shaped by hand and mirrored by the joints of the scanning needle; foot pedals; free-space hand gestures; head or eye movements; voice control; 3-6D mouse inputs; or hand-held joysticks similar to those used for video game consoles.

Lumens in the elongated member 202 (illustrated in the side view of in FIG. 20) provide at least one working channel to insert additional endoscopes, diagnostic tools, or therapeutic devices or related fluids or medications. Further, a channel may be provided to allow flow of saline for bladder scene clearing and insufflation. The saline returning from the bladder may either flow through another channel in the elongated member 202 or through to space between the elongated member 202 and the working lumen 146 of the introducer device 100. Additional channels may be provided for bending actuation cables, or the cables may be run along the outside of the scanning needle. FIG. 20 shows a preferred configuration of two working lumens 232A, 232B, two actuation channels 234A, 234B, and one saline flush channel 236.

The two working lumens 232A, 232B may be used for stereo visualization, or for different imaging modalities (e.g. one for multimodal reflectance and fluorescence forward viewing and the other for a diagnostic OCT device). One of the endoscopes may by removed during operation and replaced with another therapeutic or diagnostic device while maintaining visual observation via the remaining endoscope. Another cable (along with corresponding lumen and cable steering mechanism (described in additional detail below) may be run through the center of the elongated member 202 and terminated at the very end of the elongated member 202. This center cable, with sufficient loading tension, will compress slightly and provide a counter force to external forces, thus maintaining the current bending angles at the time of activation. This added stiffness will prevent unwanted bending and help in maintaining a steady position during visual inspection, diagnosis, or therapy.

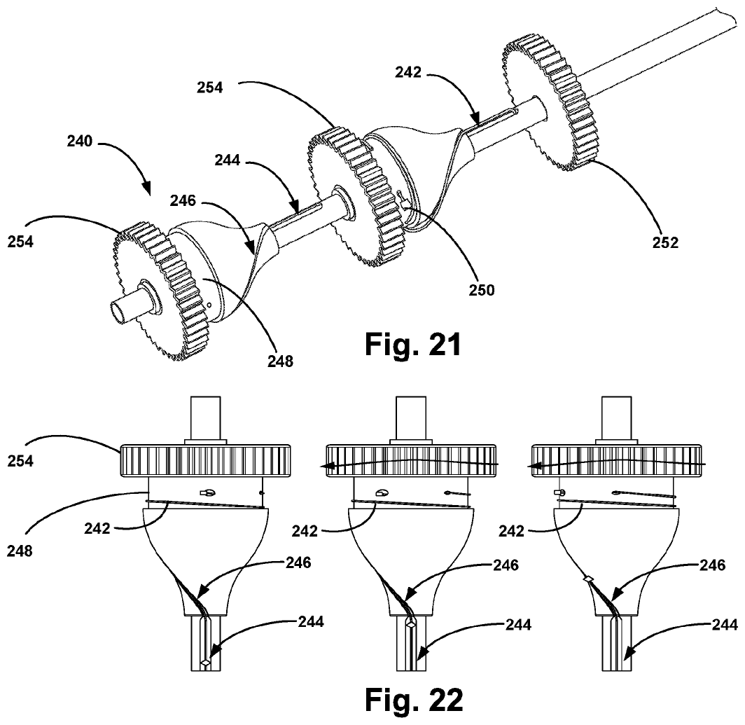

The elongated member 202 may a second linear motion mechanism 238 configured to translate the elongated member 202 within the working lumen 146 of the introducer device 100 in the direction towards the leading edge 132 of the sheath 118. In addition, the second linear motion mechanism 238 may be configured to rotate the elongated member is 202 as well as translate the elongated member 202 within the working lumen 146. In particular, the actuation of the bending of the elongated member 202 may require linear pulling of a cable along the axis of the elongated member 202. One way that this can be achieved is with gear twisters 240 as seen in FIGS. 21-22. Each steering cable 242 leaves the needle through a window 244 cut into the side of the elongated member 202. The cable 242 then enters a redirecting channel 246 that takes the cable 242 to a new path that is concentric with the axis of the elongated member 202 at an equal or similar radius to the cable capstan 248. The cable 242 wraps around the cable capstan 248 and is affixed to the cable capstan 248, either by threading the cable 242 through the cable capstan 248 and affixing a crimped end piece 250, affixing the cable 242 or an end piece 250 to the cable capstan 248, or any other method of cable fixing. A removable or latching tension maintaining device (not shown) may be used to lock the cable capstan 248 in place after manufacturer assembly to aid in device setup. This may take the form of a pin or latch. The redirecting channel 246 is affixed to the elongated member 202 so that the redirecting channel 246 always lines up with the window 244. This is one of the innovations that allows for infinite rotation of the elongated member 202 while allowing control of the bending. FIG. 22 illustrates the travel of the cable 242 as the bending gears 254 rotate.

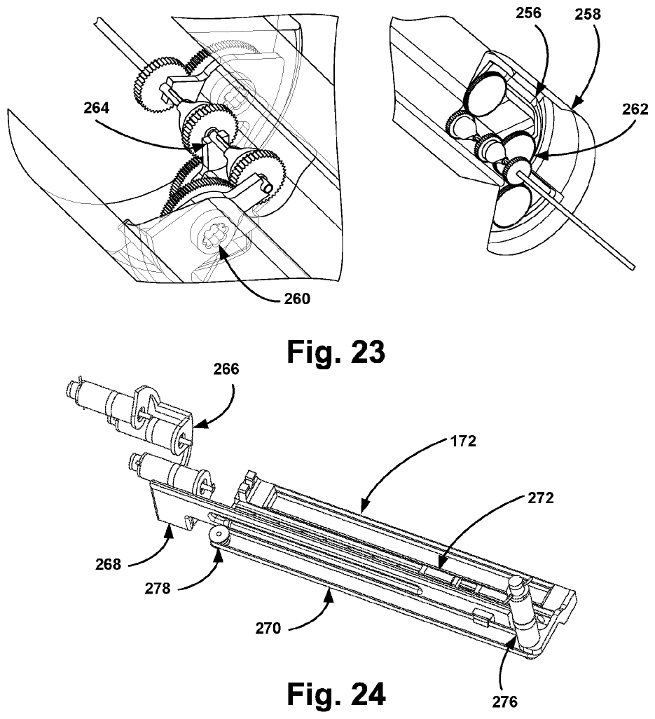

Each of the bending DOFs and the twisting of the elongated member 202 are actuated using gears or friction wheels that interface with mating components that are part of a sterile barrier, as shown in FIG. 23. The twist gear 252 is directly fixed to the elongated member 202. The bending gears 254 are fixed to the cable capstan 248 (or they are made as a single piece as shown) and allowed to rotate freely around the elongated member 202 axis. In order to maintain a constant bend angle during twist, the gears of the bending DOFs need to be spun at the same rate and the same amount. Likewise, to change a bending DOF angle while twisting, the twisting needs to be added to the control input of the bending DOF.

The steering interface 256 section of the sterile barrier is defined by one or several structures that hold mechanical interfaces between the sterile and non-sterile sides of a sterile drape 258 for each of the needle steering DOFs. The sterile drape is affixed to the perimeter of this structure. The non-sterile side of the structure features latching or attachment mechanisms (not shown) to fix the structure to the robot platform and actuator interfaces 260 that allow transmission of rotational or translational (not shown) power across the barrier. The sterile side of the structure includes the output side 262 of the actuator interface that interfaces with the mating gears or friction wheels of the elongated member 202. Furthermore, latches or yokes 264 are present to hold the elongated member 202 in place against the actuator interface 260 and transmit insertion forces. The latches or yokes 264 will provide a bearing surface for the elongated member 202 to spin freely.

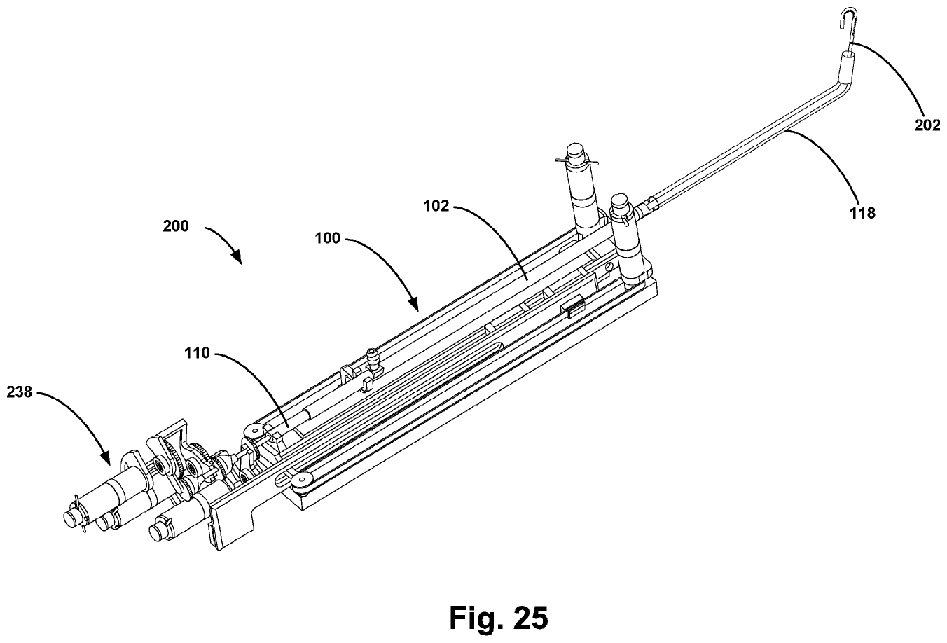

As illustrated in FIG. 24, the elongated member 202 twisting actuators are mounted to an actuator support structure 266 that is supported by the needle insertion support 268. Insertion of the needle is performed by a timing belt 270 and linear bearing 272 similar to the insertion actuation of the introducer device 100. The linear bearing 272, insertion actuator 276, and corresponding timing pulley 278 are mounted to the base plate 174 of the introducer device 100 so that the total actuation length of the elongated member 202 insertion, and thus total weight, is lowered. This is not required, and the two insertion mechanisms could be mounted independently. FIG. 25 illustrates the second linear motion mechanism 238 positioned with respect to the introducer device 100 and corresponding linear motion mechanism 172.

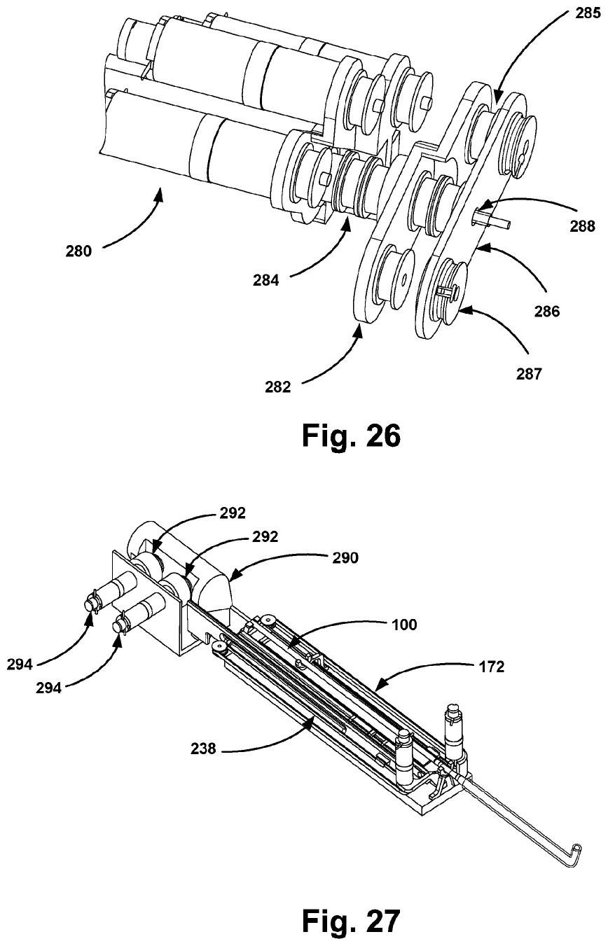

An alternate actuator layout and interface for elongated member 202 steering is shown in FIG. 26. Timing belts or gears (not shown) from each actuator 280 transmit rotation to timing pulleys or gears mounted on an assembly of concentric shafts (not shown) that transmit rotation to and through a mounting plate 282. On the other side of the mounting plate 282, power is redistributed by central timing pulleys 284 or gears to interface discs 285 with self-aligning interfaces (not shown). A sterile adapter plate 286 similar in concept to that of FIG. 24 allows transmission of power from the non-sterile actuator side to the sterile environment of the OR through sterile-plate discs 287. These sterile-plate discs 287 have an attachment mechanism to be able to attach the steering cables of the elongated member 202. The cables are routed out of the end of the elongated member 202 and around the sterile-plate discs 287. The elongated member 202 is fixed to the sterile adapter plate 286 in order to transmit twisting motion. A sterile barrier 288 needs to be extended through the needle hole in the sterile adapter plate to the opening of the inner tube 110 of the introducer device 100 in order to maintain device sterility.

In some examples, the elongated member 202 may comprise an off the shelf rigid or flexible endoscope that is inserted through the working lumen 146 of the introducer device 100. Such off the shelf devices may be controlled using adapters to robotic actuators as part of a separate robotic platform, or integrated into the introducer platform as in FIG. 27. Such an integrated system would have a similar sterile drape to the scanning needle module, but would interface directly with the manual controls of the flexible endoscope 290 using scope interface features 292 in order to actuate the bending of the endoscope. The insertion of the endoscope would be actuated using a similar linear actuation system to that of the embodiments described above. A motor 294 or other actuator would be mounted in the non-sterile side of the drape and there would be at least one for each actuation DoF of the flexible endoscope.

FIG. 28 is a block diagram of an example method for preparing and transporting a biological tissue sample for pathology. Method 300 shown in FIG. 28 presents an embodiment of a method that could be used by the introducer device 100 as described in FIGS. 1A-14 and/or the system 200 described in FIGS. 15-27, as examples. Method 300 may include one or more operations, functions, or actions as illustrated by one or more of blocks 302-306. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 300 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 302, the method 300 includes positioning the introducer device 100 (described above in relation to FIGS. 1A-14) in proximity to a body lumen of a patient. In one example, positioning the introducer device 100 in proximity to the body lumen of the patient comprises positioning the first end 106 of the outer tube 102 into the body lumen of the patient. The introducer device 100 can be configured for remote, semi-automated, or fully automated operation. At block 304, the method 300 includes applying, via the inlet port 124 of the introducer device, fluid pressure to the space 126 between the outer surface 128 of the inner tube 110, the inner surface 130 of the outer tube 102, and the sheath 118. At block 306, the method 300 includes translating the inner tube 110 from the first position to the second position to thereby cause the sheath 118 to evert from the retracted positioned within the first lumen 104 of the outer tube 102 to the extended position extending from the first end 106 of the outer tube 102 until the leading edge 132 of the sheath 118 reaches a target anatomy in the patient.

In one example, the method 300 further includes biasing, via the one or more steering wires 134 of the introducer device 100, the direction of the leading edge 132 of the sheath 118 as the sheath 118 everts from the retracted position to the extended position to direct the leading edge 132 of the sheath 118 to the target anatomy.

In another example, the method 300 further includes (i) positioning an elongated member 202 at least partially within a working lumen 146 of the introducer device 100, wherein the working lumen 146 of the introducer device is defined by a space 126 between opposing walls of the everted sheath 118, and (ii) translating the elongated member 202 through the working lumen 146 in a direction towards the leading edge 132 of the sheath 118. In one example, the elongated member 202 is translated through the working lumen 146 in the direction towards the leading edge 132 of the sheath 118 at the same speed as the sheath 118 such that there is no friction between the elongated member 202 and the sheath 118. In another example, the elongated member 202 is translated through the working lumen 146 in the direction towards the leading edge 132 of the sheath 118 at one half of a speed of the sheath 118 such that a distal end of the elongated member 202 is aligned with the leading edge 132 of the sheath 118 as the sheath 118 everts from the retracted position to the extended position.

When both the everting sheath 118 and inner cargo (e.g., elongated member 202) are extended at the same speed axially forward, the everting sheath 118 material is covering both surfaces so it moves at half the speed as the inner cargo. Thus, if you do not need the camera of the elongated member 202 to see in front to drive forward (such as situations where the surgeon has a mental model of the urethra/prostate/bladder like a nurse does when inserting a urinary catheter), then axial friction can be reduced by inserting both the sheath 118 and the elongated member 202 at the exact same speed so there is no friction from the sliding surfaces. This could be the least traumatic to the urethra or other complex small luminal structure and could be used in the bladder inspection or reinsertion of a therapeutic cystoscope after an initially much smaller diagnostic cystoscope to bring in a larger surgical tool during a minimally invasive medical procedure.

This scenario of smaller diagnostic scope for detection of a lesion and later a larger scope for the therapy procedure in the bladder is currently done, but typically the first in a clinic and the second in the hospital. Using the system 200 described herein, it is possible to do this all in the same procedure by driving a diagnostic scope in with vision to steer. If a surgical procedure is needed, then a larger scope with more tools can be inserted using the less traumatic method of having the everting sheath 118 run ahead with the larger scope starting later and then being inserted at twice speed so that the sheath 118 and larger scope arrive into bladder at the same time with almost no axial friction.

As described above, the introducer device 100 includes one or more steering wires 134 to bias the direction of the leading edge 132 of the sheath 118 as the sheath 118 everts from the retracted position to the extended position. Typically only two wires are required for single axis bending (e.g., up and down). However, there may need for dual axis bending of the sheath (e.g., left and right bending in addition to up and down bending). In such an example, the method 300 may further include rotating the elongated member 202 within the working lumen 146.

In one particular embodiment, the elongated member 202 comprises an endoscope 210 including a camera 212. In such an example, the method 300 may further include (i) capturing, via the camera 212 of the endoscope 210, one or more images of an anatomy into which the sheath 118 of the introducer device 100 is positioned, and (ii) based on the captured one or more images, biasing the direction of the leading edge 132 of the sheath 118 via the one or more steering wires 134 coupled to the sheath 118 as the sheath 118 everts from the retracted position to the extended position to direct the leading edge 132 of the sheath 118 to the target anatomy of the patient. The method 300 may further include scanning, via the camera 212 of the endoscope 210, the target anatomy of the patient. In one particular example, the body lumen comprises a urethra of the patient, and the target anatomy comprises a bladder of the patient. Other areas of the body are contemplated as well.

In another example, the method 300 further includes constructing a 3D model of bladder interior using autonomous software. In yet another example, the method 300 further includes one or more of (i) manually or autonomously identifying areas of interest for further inspection/diagnosis, (ii) rescan entire bladder/remotely investigate/semi-autonomously investigate areas of interest, (iii) replacing one channel of the endoscope with OCT/Ultrasound/Photoacoustic probe for autonomous/remote scanning/inspection to determine stage of lesions, (iv) adding contrast agents/biomarkers through Saline flush or through working lumen for lesion visualization or therapy, and (v) inserting RF Ablater/Laser Ablater/Electrocautery into working channel for remote/autonomous tumor ablation. In yet another example, the method 300 includes removing a sample of the lesion through the working lumen 126 of the introducer device 100.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. An introducer device comprising:
an outer tube defining a first lumen and having a first end and a second end;
an inner tube defining a second lumen and having a first end and a second end, wherein the first end of the inner tube is positioned in the first lumen of the outer tube, and wherein the inner tube is configured to translate within the first lumen in a direction towards the first end of the outer tube from a first position to a second position;
a sheath having a first end and a second end, wherein the first end of the sheath is coupled to the first end of the outer tube, and wherein the second end of the sheath is coupled to the first end of the inner tube;
an inlet port in fluid communication with a space defined by an area between an outer surface of the inner tube, an inner surface of the outer tube, and the sheath, wherein the sheath is configured to evert from a retracted position within the first lumen of the outer tube to an extended position extending from the first end of the outer tube, and wherein the sheath everts from the retracted position to the extended position when a leading edge of the sheath is advanced past the first end of the outer tube by applying a fluid pressure to the space via the inlet port and simultaneously translating the inner tube from the first position to the second position; and
one or more steering wires coupled to the sheath, wherein the one or more steering wires are configured to bias a direction of the leading edge of the sheath as the sheath everts from the retracted position to the extended position.

2. (canceled)

3. (canceled)

4. The introducer device of claim 1, wherein the sheath comprises an inner layer and an outer layer, and wherein the one or more steering wires are positioned between the inner layer of the sheath and the outer layer of the sheath.

5. The introducer device of claim 4, wherein a lubricant or a surfactant is positioned between the inner layer of the sheath and the outer layer of the sheath.

6. The introducer device of claim 1, further comprising:
one or more motors coupled to the one or more steering wires, wherein activation of the one or more motors adjusts a length of the one or more steering wires to thereby bias the direction of the leading edge of the sheath as the sheath everts from the retracted position to the extended position.

7. The introducer device of claim 1, further comprising:
one or more electromechanical brakes coupled to the one or more steering wires, wherein activation of the one or more brakes adjusts a length of the one or more steering wires to thereby bias the direction of the leading edge of the sheath as the sheath everts from the retracted position to the extended position.

8. The introducer device of claim 1, wherein the one or more steering wires comprise a first steering wire positioned on a first side of the sheath and a second steering wire positioned on a second side of the sheath opposite the first side.

9. (canceled)

10. The introducer device of claim 1, further comprising:
a fluid seal extending from the outer surface of the inner tube to the inner surface of the outer tube, wherein the fluid seal is positioned between the inlet port and the second end of the outer tube.

11. (canceled)

12. (canceled)

13. (canceled)

14. (canceled)

15. (canceled)

16. (canceled)

17. (canceled)

18. The introducer device of claim 1, further comprising:
a linear motion mechanism configured to translate the inner tube within the first lumen of the outer tube in the direction towards the first end of the outer tube from the first position to the second position.

19. The introducer device of claim 18, wherein the linear motion mechanism comprises:
a base plate;
a motor coupled to the base plate;
a timing belt coupled to the motor;
an inner tube mount coupled to the inner tube, wherein the timing belt is coupled to the inner tube mount such that motion of the timing belt is translated into linear motion of the inner tube; and
a linear bearing positioned between the inner tube mount and the base plate.

20. (canceled)

21. The introducer device of claim 1, wherein the sheath includes a first pressure section in fluid communication with the inlet port, and wherein the sheath further includes a second pressure section in fluid communication with a second inlet port such that a pressure in the first pressure section of the sheath can be different than a pressure in the second pressure section of the sheath to thereby further bias the direction of the leading edge of the sheath as the sheath everts from the retracted position to the extended position.

22. A system comprising:
the introducer device of claim 1;
an elongated member positioned at least partially within a working lumen of the introducer device, wherein the working lumen of the introducer device is defined by a space between opposing walls of the everted sheath;
at least one processor; and
data storage including program instructions stored thereon that when executed by the at least one processor, cause the system to:
apply, via the inlet port, fluid pressure to the space between the outer surface of the inner tube, the inner surface of the outer tube, and the sheath;
translate the inner tube from the first position to the second position to thereby cause the sheath to evert from the retracted positioned within the first lumen of the outer tube to the extended position extending from the first end of the outer tube; and
translate the elongated member through the working lumen in a direction towards the leading edge of the sheath.

23. (canceled)

24. The system of claim 22, wherein the program instructions are further executable by the at least one processor to cause the system to:
determine, based on one or more sensors positioned in the sheath, a pressure in the space between the outer surface of the inner tube, the inner surface of the outer tube, and the sheath; and
adjust, based on the determined pressure, the fluid pressure applied to the space.

25. (canceled)

26. The system of claim 22, wherein the program instructions are further executable by the at least one processor to cause the system to:
capture, via a camera of the endoscope, one or more images of an anatomy into which the sheath of the introducer device is positioned; and
based on the captured one or more images, bias the direction of the leading edge of the sheath via the one or more steering wires coupled to the sheath as the sheath everts from the retracted position to the extended position to direct the leading edge of the sheath to a target anatomy.

27. (canceled)

28. (canceled)

29. The system of claim 22, wherein the elongated member includes one or more bending slits, and wherein the elongated member further includes a cable coupled to the elongated member just distal to the one or more slits can be pulled to actuate bending of the elongated member in the direction of the slits.

30. The system of claim 22, wherein the elongated member includes a second linear motion mechanism configured to translate the elongated member within the working lumen of the introducer device in the direction towards the leading edge of the sheath.

31. (canceled)

32. (canceled)

33. (canceled)

34. A method comprising:
positioning the introducer device of claim 1 in proximity to a body lumen of a patient;
applying, via the inlet port of the introducer device, fluid pressure to the space between the outer surface of the inner tube, the inner surface of the outer tube, and the sheath; and
translating the inner tube from the first position to the second position to thereby cause the sheath to evert from the retracted positioned within the first lumen of the outer tube to the extended position extending from the first end of the outer tube until the leading edge of the sheath reaches a target anatomy in the patient.

35. The method of claim 34, further comprising:
biasing, via the one or more steering wires of the introducer device, the direction of the leading edge of the sheath as the sheath everts from the retracted position to the extended position to direct the leading edge of the sheath to the target anatomy.

36. (canceled)

37. (canceled)

38. The method of claim 34, further comprising:
positioning an elongated member at least partially within a working lumen of the introducer device, wherein the working lumen of the introducer device is defined by a space between opposing walls of the everted sheath; and
translating the elongated member through the working lumen in a direction towards the leading edge of the sheath.

39. The method of claim 38, wherein the elongated member is translated through the working lumen in the direction towards the leading edge of the sheath (i) at the same speed as the sheath such that there is no friction between the elongated member and the sheath, or (ii) at one half of a speed of the sheath such that a distal end of the elongated member is aligned with the leading edge of the sheath as the sheath everts from the retracted position to the extended position.

40. (canceled)

41. (canceled)

42. (canceled)

43. The method of claim 34, further comprising:
capturing, via a camera of the endoscope, one or more images of an anatomy into which the sheath of the introducer device is positioned; and
based on the captured one or more images, biasing the direction of the leading edge of the sheath via the one or more steering wires coupled to the sheath as the sheath everts from the retracted position to the extended position to direct the leading edge of the sheath to the target anatomy of the patient.

44. (canceled)

45. (canceled)
